(19) United States
(12) Patent Application Publication
(10) Pub. No.: US 2009/0074736 A1
(43) Pub. Date: Mar. 19, 2009

(54) IMPLANTABLE PREPARATIONS COMPRISING GLOBIN, PROCESS FOR THEIR PRODUCTION, AND USES

(76) Inventors: Jean-Louis Tayot, La Tour De Salvagny (FR); Florent Sapin, Lyon (FR); Jean Bernasconi, Asnieres (FR)

Correspondence Address: STITES & HARBISON PLLC, 1199 NORTH FAIRFAX STREET, SUITE 900, ALEXANDRIA, VA, 22314, US

(21) Appl. No.: 12/207,621

(22) Filed: Sep. 10, 2008

Related U.S. Application Data
(60) Provisional application No. 60/971,349, filed on Sep. 11, 2007.

(30) Foreign Application Priority Data
Nov. 9, 2007 (FR) 07 57501

(51) Int. Cl. A61K 35/12; A61K 38/17

(52) U.S. Cl. 424/93.71; 514/12; 424/93.72

(57) ABSTRACT

Preparations comprising, especially, globin that is insoluble at neutral pH, and therefore at physiological pH, in which the globin has been obtained from whole blood by depigmentation in a medium that extracts or dissolves the haem but leaves the globin and the other constituents of proteinic nature in a substantially undissolved state, a process for the production of those preparations, and uses, especially for the filling, healing or regeneration of tissues, especially for chronic wounds and osseous healing or regeneration.

### BACKGROUND OF THE INVENTION

The present invention relates to novel implantable preparations comprising globin, to a process for their production, and to the uses, especially therapeutic uses, of those preparations.

Implantable preparations comprising globin and uses of those preparations have already been described. For example, applications and patents FR0305700, WO2004/100934 (EP1622596), U.S. Pat. No. 6,949,625 describe preparations comprising globin that is insoluble at neutral or physiological pH. Applications FR0508392, PCT/FR2006/001880, US Application 2007/0031474 describe implantable preparations comprising a soluble or insoluble material which can be obtained from globin that has been treated, especially by chemical means, in order to become at least partially soluble at that pH. Application PCT/FR2007/000137, which has not yet been published, describes improvements to preparations comprising globin. All those applications and patents are incorporated by reference herein.

The globin employed in those documents can be of heterologous origin with respect to the treated species, but it is far preferable for it to be homologous, for example prepared from blood transfusion bags, especially bags of out-of-date erythrocytes. Particularly preferably, the globin can be autologous, obtained from blood taken from the patient himself. Preparations at neutral pH comprising globin that is insoluble at neutral pH can be prepared in non-solid and directly sterile form or a form sterilized, e.g. through beta or gamma irradiation, with excellent fluidity, permitting injection even through the finest hypodermic needles, for example in order to fill cutaneous wrinkles.

The present invention proposes to provide novel preparations, which can be administered by implantation, including injectable ones, comprising globin that are particularly suitable for certain uses, especially for surface implantations, for example for covering cutaneous wounds, such as chronic wounds, especially in diabetics, or implantations for filling large volumes, for example osseous sockets after dental extraction, or periodontal pockets or large lacunae, osseous or otherwise.

### SUMMARY OF THE INVENTION

The invention relates to preparations comprising, especially, globin that is insoluble at neutral pH, and therefore at physiological pH, in which the globin has been obtained from whole blood by depigmentation in a medium that extracts or dissolves the haem but leaves the globin and the other constituents of proteinic nature in a substantially undissolved state.

Accordingly, when the medium that has extracted or dissolved the haem is removed, it is possible to obtain, for example by filtration or centrifugation, a material that comprises globin that is insoluble at physiological pH and that also comprises globin that is soluble at physiological pH and, in smaller amounts, plasma proteins, especially albumin, alpha-, beta- and gamma-globulins, and which may further contain coagulation factors and platelet factors, which factors may also be added, if needed.

That active proteinic material derived from blood and decolored, called “proteinic material”, can be used in the form selected from the group consisting of a paste and suspension in a physiologically acceptable carrier.

It can also be brought into the more or less dry state, for example by a method selected from the group consisting of desiccation and lyophilization, and can be in a form selected from the group consisting of a powder, granules, a gel, a film, a sponge and a preformed solid implant, especially in the forms described in the applications and patents listed hereinbefore.

The preparation can be, especially, in an acidic state or in a neutral state.

The material can be in association with supports, especially implantable supports, for example various dressings, surgical wires or surgical textiles, especially the supports described in the applications and patents listed hereinbefore.

It can also be in association, in the preparation, with other substances or active ingredients, especially those described in the applications and patents listed hereinbefore. For example, it can be in association with the following active substances: antibiotics, antimicrobial products such as silver salts, homologous or autologous fibrin glue, biocompatible chemical adhesives.

Before or at the time of its use, it can be combined with various cells, including the stem cells of the blood lines or of other organs isolated from the same patient or from a different donor, with a view to grafting those cells for cell therapy applications.

For example, the preparation can comprise leucocytes and/or platelets, preferably leucocytes and/or platelets from the whole blood from which the preparation has been obtained.

Finally, the preparation can comprise auxiliary substances, for example lubricating agents, especially those described in the applications and patents listed hereinbefore: among those there may advantageously be mentioned the following: sodium hyaluronate, gelatin, oxidized cellulose, cellulose esters, polyethylene glycol, glycerine, or mixtures thereof. The mineral supports also constitute a family of auxiliary substances, especially calcium phosphates, hydroxy apatite, which can be used for bone filling.

Preferably, the concentration of the above-mentioned proteinic material in the preparation, in the absence of any support, is from 2 or 2.5% to 100%, globin itself preferably forming the substantial majority of said proteinic material. Particularly preferably, the percentage of insoluble and soluble globin represents more than 60% of the total proteinic material. Globin that is insoluble at physiological pH represents a solid support to which the cells can become attached and multiply, constituting a filling material which accelerates the healing process of wounds, of organs capable of self-regeneration or healing, or the filling of cavities or other voids.

The globin can be of heterologous origin with respect to the treated species, but it is far preferable for it to be homologous, for example prepared from blood transfusion bags, especially bags of out-of-date erythrocytes. Particularly preferably, the globin can be autologous, obtained from blood taken from the patient himself.

The invention relates also to a process for obtaining those preparations, in which process whole blood, preferably defrosted or hemolyzed whole blood, is treated with, where appropriate, the cell elements or cell debris in suspension in a medium that extracts or dissolves the haem but leaves the globin and the other constituents of proteinic nature in a substantially undissolved state, and the pigmented medium is then removed in order to obtain the globin together with other plasma proteins.

In a particular embodiment, the leucocytes and/or platelets are first separated from the blood in a sterile manner and are stored separately so that they do not undergo the haem-extracting treatment, which is aggressive towards those cells. These cells can finally be mixed again with the proteinic material comprising globin. The whole yields a cellularized filling material, based on globin as insoluble support, which can have many medical applications in the healing field, by promoting the elimination of bacteria from the wound, but also in the field of grafts and cell therapy.

Preferably, the medium for extraction of the haem is an acetonic, preferably acidic, medium, for example containing 99 volumes of acetone and 1 volume of concentrated hydrochloric acid (12N). The defrosted and hemolyzed blood can preferably be subjected to prior oxidation, which permits the conversion of hemoglobin into methemoglobin in order to improve the subsequent decoloration. The oxidation can be carried out, for example, in the presence of sodium nitrite and of glycocoll at neutral pH. The oxidized blood is then poured slowly and with vigorous stirring into an acidic acetonic solution, which brings about the immediate extraction of the haem and the simultaneous precipitation of the blood proteins. The precipitated proteinic material is preferably washed with several volumes of acetone before being dried in vacuo in order to remove any residual traces of acetone. At this stage, the proteinic material is in the form of an acidic powder.

Neutralization of that powder can then be carried out in order to optimize its biocompatibility with the tissues and wounds to be treated. The acidic material or powder is brought back into aqueous solution and then neutralized, preferably by addition of an alkaline or buffered solution of the phosphate or carbonate type. A neutral powder can then be obtained by lyophilization or acetonic precipitation. It can optionally be resuspended by addition of a physiological saline solution in order to prepare a more or less thick paste.

All the steps of the process can be carried out in the same sterile environment, for example under a chemical hood situated in a laboratory or a sterile room. In such case, it is preferred to use materials and products which are first sterilized. Such materials and products can be used as a kit which is specially prepared for each blood sample.

In addition, the resulting globin material, in the moist or dry state, preferably in the form of a finished, packaged preparation, can be directly sterile, or sterilized by beta or gamma radiation at a dose of from 5 to 30 kGrays, if necessary in frozen form, for example in dry ice.

The preparations according to the invention have great qualities of integration into the receiving tissue media. The implanted preparation is readily colonized by the locally pertinent cells, for example fibroblasts, osteoblasts, chondroblasts, stem cells, which are able to differentiate to form a new tissue which gradually replaces the mass of implanted insoluble globin. Accordingly, the preparations according to the invention can be used in the healing of surgical tissue wounds, of chronic wounds, for example in diabetic patients, and can facilitate the formation of a vascularized dermal and epidermal tissue.

They can be used especially to assist the healing or reconstitution of parts of organs or of osseous tissues, for example in open fracture cavities, dental extraction sockets, or spaces adjacent to dental implants, suitably filled with the preparation, or in periodontal pockets.

In general, the preparations according to the invention can be used for the filling, healing or regeneration of tissues, especially of connective or osseous tissues.

It is generally preferred, within that context, to use dry forms, especially preparations in the form of powder or granules, which facilitates the penetration of the implanted preparation through the serosa of the patient.

The invention accordingly relates also to a treatment or filling method, especially for healing, in which a therapeutically effective amount of a preparation according to the invention is implanted into or onto a tissue of a patient who has the need therefor.

The invention relates also to the use of said proteinic material in the production of preparations for the purpose selected from the group consisting of treatment, filling, and for the regeneration of tissues, especially for the healing of tissues, especially of connective or osseous tissues.

Other advantages and characteristics of the invention will become apparent upon reading the following description, which is given by way of non-limiting examples.

### DETAILED DESCRIPTION OF THE PREFERRED EMBODIMENTS

### EXAMPLE 1

### Preparation of an Acidic Acetonic Powder Comprising Globin and Blood Proteins

30 ml of human blood are taken from a bag of blood from a voluntary donor and are immediately placed in a freezer at −20° C. After defrosting, 10 ml of distilled water are added to the blood in order to complete the hemolysis of the red blood cells. 0.8 ml of 1M glycine and then 0.4 ml of 1M sodium nitrite are added. After oxidation of the hemoglobin to methemoglobin for 1 hour, the blood assumes a black colour. If the oxidation is not carried out, the decoloration of the final powder is less effective, which can hinder its acceptance by the patient. The solution of oxidized blood is then added dropwise, with vigorous stirring, to 400 ml of acetone (10 volumes) containing 4 ml of 12N concentrated hydrochloric acid. The black pigment of the haem is extracted in acetone, while the globin is immediately decolored and precipitated with the other blood proteins. The suspension is then filtered through a rectangular textile filtration membrane having a porosity of about 1 micron, which is rolled up along its length to form a tube, the two sides of which are bonded together (for example: tubes having an inside diameter of 20 mm, bonded by ultrasound, made of SEFAR NITEX 03-1/1 fabric). One end of the tube is connected to a funnel for introducing the suspension to be filtered, while the other end is closed off by a clip. The proteinic precipitate accumulates in the tube, while the pigmented acetonic solution is readily filtered and can be removed into a retention container before being evacuated for recycling of the acetone. At the end of the filtration, the acidic precipitate is resuspended in 200 ml of acetone and then filtered again through the same tube in order to remove residual traces of pigments. Washing is repeated a second time in order to complete the decoloration of the acidic proteinic precipitate. In order to improve the decoloration still further, the acidic precipitate can be redissolved in 100 ml of sterile distilled water and the resulting solution can then be poured into 10 volumes of acidic acetone again in order to remove final traces of pigments and reprecipitate the globin and the plasma proteins. The operations of filtration and washing with acetone are reproduced as in the first treatment. The final acidic proteinic precipitate is then spread out in a small thickness under a stream of air in a sterile hood with laminar flow. There are finally obtained 4.25 g of an acidic proteinic powder which is white to bright ocher in color and comprises globin and plasma and platelet proteins, including in particular albumin, alpha-, beta- and gamma-globulins, and coagulation, cell multiplication and healing factors. These various proteins can readily be displayed by the common technique of polyacrylamide gel electrophoresis in the presence of sodium dodecyl sulfate.

### EXAMPLE 2

### Preparation of a Neutral Lyophilized Powder Comprising Globin and Blood Proteins

The powder of Example 1 is prepared according to the described process. The powder is then taken up in 100 ml of sterile distilled water, neutralized to pH 7 by addition of 1M sodium hydroxide solution, and then lyophilized. The resulting neutral powder is finely divided and flows readily. It is white to bright ocher in color. It is sterilized in its final packaging by beta or gamma radiation at a dose of from 5 to 30 kGrays.

### EXAMPLE 3

### Preparation of a Neutral Acetonic Powder Comprising Globin and Blood Proteins

The powder of Example 1 is prepared according to the described process. The powder is then taken up in 100 ml of sterile distilled water and then neutralized to pH 7 by addition of 1M sodium hydroxide solution. The neutral suspension is then poured slowly into 1000 ml (10 volumes) of acetone, with vigorous stirring. All the proteins precipitate immediately and the precipitate is collected by filtration through the same type of filtering tube as that used in Example 1. The proteinic precipitate is washed twice with 200 ml of acetone, harvested after decantation and then finely divided with a spatula and dried in a thin layer under a stream of air in a sterile hood with laminar flow. The resulting neutral powder is finely divided and flows readily. It is white to bright ocher in color. It is sterilized in its final packaging by beta or gamma radiation at a dose of from 5 to 30 kGrays.

### EXAMPLE 4

### Preparation of a Neutral Paste Comprising Globin and Blood Proteins

The neutral acetonic powder of Example 3 is prepared without being irradiated. 5 g of powder are taken up in 25 ml of a physiological saline solution of the PBS or Ringer type, and the resulting paste is distributed into its final packaging (for example in a flexible tube). The paste is frozen in dry ice at −80° C. and irradiated in frozen form with beta- or gamma-radiation. After defrosting, the sterility of the tubes of proteinic paste is checked by microbiological tests. There are finally obtained 30 g of a neutral proteinic paste, bright ocher in color, which comprises globin and the plasma and platelet proteins, including in particular albumin, alpha-, beta- and gamma-globulins, and coagulation, cell multiplication and healing factors. These various proteins can readily be displayed by the common technique of polyacrylamide gel electrophoresis in the presence of sodium dodecyl sulfate. Irradiation in frozen form does not bring about any significant modification of the electrophoretic profile of the proteins.

### EXAMPLE 5

### Preparation of a Directly Sterile Globin Powder or Paste or Globin Based Material

The liquid or solid globin materials of Examples 1 to 4 are prepared under a sterile air flow hood located in a sterile or particle free laboratory, which air is constantly renewed by sterile filtration, according to the electronical or pharmaceutical industry standards.

All the required materials are prepared in advance, by the operations of washing and packaging into one or several containers, preferably into double plastic pouches, which are then sterilized by autoclave or by irradiation. The globin paste manufacturing set, comprising a flexible filter bag or tubular hose, for instance with a porosity of about one micron, connected or designed to be connected to an upper funnel, and closed or designed to be closed at its lower part via a clip or grip, for instance the set described in Example 1, and preferably assembled in advance, is an essential part of this set. Thus one or several sterilized kits are obtained, which are specifically adapted for the manufacturing of a globin paste, or more generally a globin based material. The final globin preparation is directly sterile and does not need any final sterilization by irradiation. This simplifies and greatly decreases the preparation time, including for the autologous globin preparations, which can be manufactured in a laboratory close to the patient to be treated in the hospital or the clinic.

This method and means for preparing purified globin can also be used for the preparation methods using red cells separated from the plasma, described in French Patent FR 0305700 and U.S. Pat. No. 6,949,652, which are herein incorporated by reference.

In addition to said filtrating set, the individual kit may be provided in two exemplaries, and preferably may contain, in the same sterile pouch or in a separate one, one or several items, such as centrifuge tubes, magnetical stirring bars, pipets for pH adjustment, vials or syringes for collecting the globin powder, syringes for collecting the globin paste, homogenisation connectors and caps for the globin syringes, membrane filters all adaptable to Luer locked syringes. Bottles containing the required reactants or buffers may be incorporated in the individual kit or be separately sterilized to be associated with the kit elements at the time of the sterile manufacturing of the globin.

### EXAMPLE 6

### Preparation of a Neutral Paste Comprising Globin and Blood Proteins, Enriched with the Donor's Leucocytes

The neutral acetonic powder comprising globin of Example 3 or 5 is prepared according to the described process, except for the initial treatment of the blood.

The freshly obtained blood sample can, for example, be centrifuged at 3000 rpm for 20 minutes in a sterile cup. The intermediate leucocyte layer is aspirated by means of a pipette. The leucocytes of the sample are washed with a buffered physiological solution of the Ringer or PBS type and are then brought into the presence of preserving agents that are already known, especially for freezing at −80° C. This leucocyte suspension can ultimately be remixed with the globin paste.

The plasma layer is then mixed with the pellet of red blood cells, and the whole is then frozen. After defrosting, the hemolyzed suspension can be treated according to Example 1. The neutral acetonic powder comprising globin is then prepared according to Example 3. Finally, the globin powder is sterilized, if needed, by irradiation at 30 kGrays and can be taken up in a minimum volume of physiological solution when it is mixed with the sterile leucocytes. The cellularized globin-based proteinic material can be used for healing applications or for applications of grafting stem cells of the blood line, in the treatment of hemopathies or for cell therapy applications.

### EXAMPLE 7

### Healing Properties of the Material Comprising Porcine Globin and Blood Proteins

A neutral powder or paste preparation comprising globin and blood proteins is prepared from pig's blood according to any one of Examples 2 to 6.

An experiment on deep rectangular cutaneous wounds 2×3 cm in size which have been created in the pig allows the value of this homologous proteinic material in the healing process to be checked. The volume of the wound is filled with the porcine proteinic material and the wounds are then covered with a non-adherent dressing of the Tegaderm® type. The pig's body is then covered with a tight dressing which protects the wounds from any external contamination. Every 3 to 4 days, the wounds are cleaned with physiological water and, when a cavity is still present, the same proteinic filling material is applied, until a granulation tissue which fills the initial wound is created.

The presence of a soluble portion in the powder permits the aspiration and drainage of the physiological fluids, the “exsudates” of the wound, which dissolve the soluble globin, hydrate the insoluble globin powder and liberate the other constituents which are important for the cells and the development of the granulation tissue. The interstitial space thus freed permits optimum migration and cell colonization of the implant. Those fluids and the cells migrate and are adsorbed on the granules of insoluble globin and promote rapid filling by the granulation tissue. That filling agent provides virtually the majority of the elements of the blood clot. It is perfectly tolerated and degrades in less than two weeks. It significantly reduces contraction by the sides of the wound and promotes harmonious healing in less than four weeks, characterized by complete epithelialization of good quality.

### EXAMPLE 8

### Medical Applications of the Material Comprising Globin and Proteins of Human Blood

The powder obtained according to any one of Examples 1 to 5 is finely divided and flows readily, which facilitates its local application to external or internal wounds with the aid of vials, sachets or other conventional applicators, or by pulverization. The paste of Examples 5 and 6 can also be used in contact with very sensitive cutaneous wounds, for which a dry product would risk being a little more irritating. The paste can be applied by injection with the aid of syringes into internal wounds or cavities, or superficially by manual pressure on tubes. The preparations according to the invention can be used for filling wrinkles, other cutaneous flaws or deficient sphincters. They can be used for the healing of internal surgical wounds, of parts of organs, of chronic wounds of cutaneous tissues, for example in diabetic patients or patients affected by arterial or venous disorders which create poor local vascularization of the limbs, or in patients affected by eschars. The concentrate of globin and blood proteins locally provides certain healing blood factors that normal irrigation of the tissues no longer provides in such patients. Furthermore, the particles of insoluble globin promote and accelerate the formation of a specific vascularized connective, dermal or epidermal tissue. The implanted preparation is readily colonized by the locally pertinent cells, such as fibroblasts, which are able to differentiate in order to synthesize collagen fibrils and form a new tissue, which gradually replaces the implanted proteinic material. The preparations can also be used for aiding the regeneration of a granulation tissue on deep burn wounds after excision of the necrotic tissues. This step is important prior to the grafting of sheets of epithelium cultivated in vitro.

The preparations can also be used for assisting the healing or the reconstitution of osseous or cartilage tissues, for example in the cavities of open fractures, dental extraction sockets, suitably filled with the material, or in periodontal pockets. The proteinic material facilitates the migration, attachment and differentiation of the osteoblasts or chondrocytes and, more generally, of the stem cells present in or added to the preparation. It permits more rapid filling and tissue generation.

What is claimed is:

1. A preparation comprising globin that is insoluble at physiological pH and other blood constituents of proteinic nature, in which the globin has been obtained from whole blood by depigmentation in a medium that extracts or dissolves the haem but leaves the globin and said other constituents of proteinic nature in a substantially undissolved state.

2. The preparation as claimed in claim 1, comprising a material that comprises globin that is insoluble at physiological pH with globin that is soluble at physiological pH and, in smaller amounts, plasma proteins, especially albumin, coagulation factors and platelet factors.

3. The preparation as claimed in claim 1 wherein said whole blood is human blood.

4. The preparation as claimed in claim 1 in an acidic state.

5. The preparation as claimed in claim 1 in a neutral state.

6. The preparation as claimed in claim 1 comprising leucocytes and/or platelets.

7. The preparation as claimed in claim 6, in which the leucocytes and/or platelets are from the whole blood from which the preparation has been obtained.

8. The preparation as claimed in claim 1, in a form selected from the group consisting of a paste and a suspension in a physiologically acceptable carrier.

9. The preparation as claimed in claim 1, in a form selected from the group consisting of a powder, granules, a gel, a film, a sponge and a preformed solid implant.

10. The preparation as claimed in claim 1, in association with a support, especially an implantable support.

11. The preparation as claimed in claim 1, comprising at least one other substance or active ingredient.

12. The preparation as claimed in claim 1, comprising at least one auxiliary substance.

13. The preparation as claimed in claim 1, in which the concentration of the above-mentioned material in the preparation, in the absence of any support, is from 2 or 2.5% to 100%.

14. The preparation as claimed in claim 13, in which the globin forms the substantial majority of said material.

15. A process for obtaining the preparation as claimed in claim 1, in which whole blood, preferably defrosted or hemolyzed whole blood, is treated in a medium that extracts or dissolves the haem but leaves the globin and the other constituents of proteinic nature in a substantially undissolved state, and then the pigmented medium is removed to yield the globin together with other blood proteins.

16. The process as claimed in claim 15, in which the whole blood is treated in an acidic medium.

17. The process as claimed in claim 16, in which an acidic solution of the material comprising globin is subsequently neutralized.

18. The process as claimed in claim 17, in which a substantially solid preparation is produced by a method selected from lyophilizing or precipitating in acetone the suspension obtained by neutralization.

19. The process as claimed in claim 15, in which the leucocytes and/or platelets are separated from said whole blood before the treatment in said medium that extracts or dissolves the haem, and, optionally, the separated leucocytes and/or platelets are added to the preparation again.

20. A kit comprising, in a sterile container a flexible filtration hose connected or connectable to an upper funnel, and closed or closable at its lower part via a clip or a grip.

21. A method for treating, filling or regenerating, including healing, a tissue, comprising the step of implanting into or onto a tissue of a patient in need thereof, of an amount of a preparation according to claim 1.

22. A method according to claim 21 wherein said tissue is a conjunctive or bone tissue.

23. A method according to claim 21 for the healing of surgical tissue wounds, of chronic wounds, including in diabetic patients, facilitating the formation of a vascularized dermal and epidermal tissue, healing or reconstitution of parts of organs or of osseous tissues, including in open fracture cavities, dental extraction sockets, or spaces adjacent to dental implants, suitably filled with the preparation, or in periodontal pockets.
